(19) United States
(12) Patent Application Publication
(10) Pub. No.: US 2006/0134046 A1
(43) Pub. Date: Jun. 22, 2006

(54) Skin nourishing and moisturizing cream

(76) Inventors: Ibrahim Hanna, Kearny, NJ (US); Madiha Elaguizi, Kearny, NJ (US); John Hanna, Kearny, NJ (US); Haidy Hanna, Kearny, NJ (US)

Correspondence Address: HANNA IBRAHIM, 4 STUYVESANT AVENUE, KEARNY, NJ, 07032, US

(21) Appl. No.: 11/018,478

(22) Filed: Dec. 21, 2004

(51) Int. Cl. A61K 8/73; A61K 8/97; A61K 36/889; A61K 36/736

(52) U.S. Cl. 424/70.13; 424/74; 424/735; 424/765; 424/727

(57) ABSTRACT

A very rich, all natural nourishing, super-moisturizing and anti-aging cream. The composition comprises a water in oil emulsion blended to a creamy texture. The cream is produced by blending specific quantities of spermacetti, anhydrous lanolin, pure beeswax, sweet almond oil and coconut oil with glycerin, rose water, hydrolysed collagen, hyaluronic acid and distilled water at a certain temperature (70-75 C. degree) then mixing at high speed. The product contains no alcohol or preservatives

### BACKGROUND OF THE INVENTION

The present invention relates to skin care preparations and in particular to a very rich, all natural nourishing anti-dryness and anti-aging cream.

The major function of skin is to provide a barrier between the body and the outside environment. The outermost layer of the epidermis stratum corneum is made up of sheets of dead keratinized cells that serve as a water proof barrier to the external danger environment. Also melanocytes produce brown pigment, which helps protect the body from ultraviolet light. The middle layer, the dermis, provides a tough, flexible foundation for the epidermis. In the dermis, sweat glands and blood vessels help to regulate body temperature, nerve endings send sensations to the brain, also oil glands produce sebum which help to moisturize the skin.

Lack of moisture in the lower layer of the skin results in a wrinkled and aged look. Lack of moisture of the sratum corneum is more noticeable and marked by roughness, increased flakiness and in more severe cases, cracks and actual peeling. The stratum corneum remains soft and pliable only as long as the moisture content exceeds 10%. Below this the skin becomes hard and brittle and develops an opacity.

In the lower layer of the skin, degenerative changes occur with age whereby not only moisture is lacking but also a major amount of lipoidal or fatty substances.

The skin is subject to deterioration through dermatological disorders, environmental abuse (wind, air conditioning and central heating) or through the normal aging process (chronoaging) which may be accelerated by skin exposure to sun (photoaging).

A constant need exists for nourishing the skin to prevent and treat aging process that appear as lines, wrinkles and dry skin. The pharmaceutical industry has long suggested the use of natural oils and avoidance of artificial ingredients for the treatment of such problems.

U.S. Pat. No. 4,986,986 (Roth) discloses a moisturizing lotion based on a blend of peanut oil, rose water, olive oil, anhydrous lanolin and natural lemon oil while specifically avoiding artificial ingredients, preservatives, emulsifiers or alcohol.

U.S. Pat. No. 5,002,974 (Geria) discloses a moisturizing composition for treating dry skin based on an oil phase in aqueous phase where surface active agents assist in forming a stable oil in water emulsion.

U.S. Pat. No. 1,631,384 (Richmond) discloses a skin lotion containing pure citrous pectin (e.g. lemon pectin) to which other materials may be added including glycerin, oils, lanolin, almond oil, stearic acid and the like.

The composition of the nourishing moisturizing and anti-aging cream in claim 1 consists of an oil phase (claim 3) and a water phase (claim 5). The composition of the oil phase of the nourishing cream consists of five natural ingredients: spermacetti, anhydrous lanolin, pure beeswax, sweet almond oil and coconut oil.

These ingredients are particularly effective in restoring natural oils to the skin and smoothing aging wrinkles, as they potentiate each other actions. Also they produce a fast and long lasting nourishing effect.

The composition of the water phase of the nourishing cream consists of glycerin, rose water, hydrolysed collagen and hyaluronic acid. These ingredients have a combined strong moisturizing effect on the skin and they supplement it with the natural substances (collagen and hyaluronic acid) which become depleted by age.

### REFERENCES CITED

### US Patent Documents

| | | |
|---|---|---|
| 5,972,362 | OCTOBER, 1999 | Wenker |
| 4,936,986 | JUNE, 1990 | Roth |
| 5,002,974 | MARCH, 1991 | Geria |
| 5,242,952 | JUNE, 1992 | Tritsarolis |
| 1,631,384 | JUNE, 1927 | Richmond |

### BRIEF SUMMARY OF THE INVENTION

Briefly, the invention pertains to a very rich nourishing, moisturizing and anti-aging cream comprising by volume 3% spermacetti, 10% anhydrous lanolin, 3% pure beeswax, 15% sweet almond oil, 15% coconut oil, 7.5% glycerin, 12.5% rose water, 2% hydrolysed collagen, 2% hyaluronic acid and 30% distilled water.

Advantage of this invention. The presence of these nine active natural ingredients together provide a very rich nourishing, moisturizing and anti-aging cream giving a faster and long lasting effect on the skin.

### DETAILED DESCRIPTION OF THE INVENTION

The nourishing cream of this invention comprises an oil phase and a water phase processed together in a stepwise process to produce a uniform mixture of the components: water in oil emulsion.

Refering to the first component of the skin nourishing cream of this invention spermacetti: it is a white waxy substance refined from the sperm oil obtained from cavities in the head of the sperm whale. It consists essentially of ethereal salts of palmitic acid with ethal and other hydrocarbon bases.

The second component of the skin nourishing cream of this invention comprises anhydrous lanolin. It is widely recognized as an excellent emollient and is readily absorbed by the stratum corneum where it reduces excess moisture loss and restores softness and elasticity to dry skin.

It also has a powerful water-oil emulsifying properties and adds water resistant properties to skin care products. (Melting temperature 36-44 C. degree).

The third component of the skin nourishing cream of this invention comprises pure beeswax, which is a by-product secreted from the wax glands on worker bees abdomen. Beeswax is rich in vitamin A necessary for healthy skin tissue. It is added to this composition to influence its consistency and to form a barrier on the skin which empedes water loss. Hand cream containing beeswax is specially good during winter months for dry skin.

The fourth component of the skin nourishing cream of this invention is sweet almond oil. It is an odorless oil derived from pressed almonds. It nourishes the skin with mono and polyunsaturated fatty acids, antioxidants and naturally occuring vitamins A, B1, B2, B6 and vitamin E. Sweet almond oil is easily absorbed into the skin leaving it soft, smooth thus promoting a younger looking complexion, it also relieves skin itch and dryness.

The fifth component of the skin nourishing cream of this invention is coconut oil. It is a triglyceride of lauric acid (principal fatty acid) a saturated fat consisting primarily of medium chain fatty acids, it is not easily oxidized and does not cause harmful free radical damage like polyunsaturated vegetable oils. Also coconut oil has wonderful antioxidant properties that protect the skin from free radical damage that may be caused by exposure to the sun.

The sixth component of the skin nourishing cream of this invention comprises glycerin which is a humectant derived from vegetable fatty acids, drawing vital moisture to the skin to insure flexibility and resilience. Glycerin penetrates the skin to help moisturize from the inside out. It softens the skin and is best for people with dry or sensitive skin.

The seventh component of the skin nourishing cream of this invention comprises rose water or floral water. It stimulates and tones the skin. It is balancing for both dry and oily skin types as it controls and balances sebum production while having antibacterial properties. Its pH balancing and restoring effect helps tighten pores.

The eighth component of the skin nourishing cream of this invention comprises hydrolysed collagen. The collagens, a family of closely related proteins, are the main fibrillar component of the connective tissues and the major extracellular proteins of the human body. As a result of aging, exposure to various climatic conditions such as sun and wind, in addition to loss of moisture in the epidermal layer of the skin, loss of elasticity and skin tone occur through degradation of collagen and its gradual replacement by the weaker glycoaminoglycans GAGS which are less thin and less elastic. Also photo-aging involves damage to the collagen fibers of the skin.

A wrinkle, very simply, is caused by the reduction of collagen. So the use of hydrolysed collagen in this skin nourishing cream has a major function as a highly efficient natural agent for the retention and enhancement of skin moisture and elasticity. Also human collagen plays an important role in the wound healing process.

The ninth component of the nourishing cream of this invention is Hyaluronic acid. Hyaluronic acid is a special protein, mucopolysacharide, occurring naturally through the human body. Hyaluronic acid is an excellent moisturizer as it is able to hold more water than any other natural substance. It is one of the most hydrophillic molecules in nature. Hyaluronic acid restores elasticity and hydration to the skin increasing smoothness and softening and decreasing wrinkles and aging lines, so giving it a more youthful appearance.

Hyaluronic acid has been scientifically proven to stimulate new cell growth. Hyaluronic acid acts also as a powerful antioxidant protecting against repetetive wear strain and chronic degeneration. It provides structural integrity to the cellular matrix that inhibits bacteria and viruses from reaching cells. With advancing age, there is a decrease in synthesis of hyaluronic acid resulting in thinning and decreased moisture in the skin, leaving it dry and wrinkled. So supplementation with hyaluronic acid is crucial.

Method

The nourishing, moisturizing and anti-aging cream of this invention is prepared as follows:

1—All of the oil phase ingredients: 3% spermacetti, 10% anhydrous lanolin, 3% pure beeswax, 15% sweet almond oil and 15% coconut oil, are put in a container heated in a hot water bath, till the temperature of the mixture reaches 70 to 75 C. degree.

2—All of the water phase ingredients: 7.5% glycerin, 12.5% rose water, 2% hydrolysed collagen, 2% hyaluronic acid and 30% distilled water are put separately in a container heated in a hot water bath, till the temperature of the mixture reaches 70 to 75 C. degree.

3—The hot water phase is slowly added to the hot oil phase in the following manner:

With the oil phase at 75 C. degree, the blender or mixer is started and hot water phase is added slowly while stirring.

4—The mixture (while in liquid state) is vigorously blended at a high speed to insure complete emulsifiquation.

5—Blending is continued till the temperature of the mixture decreases to 20-25 C. degree.

6—The final product is creamy.

What we claim as our invention is:

1. A skin nourishing moisturizing and anti-aging cream consisting essentially of oil phase and water phase.
The oil phase of skin nourishing cream consisting essentially of:
3% by volume Spermacetti.
10% by volume anhydrous Lanolin
3% by volume pure Beeswax.
15% by volume sweet Almond oil.
15% by volume Coconut oil.
Based on the volume of the composition.
The water phase of skin nourishing cream consisting essentially of:
7.5% by volume Glycerin.
12.5% by volume Rose water.
2% by volume hydrolysed Collagen.
2% by volume Hyaluronic acid.
30% by volume Distilled water.
Based on the volume of the composition.
